(19) United States
(12) Patent Application Publication
(10) Pub. No.: US 2026/0294773 A1
(43) Pub. Date: Oct. 1, 2026

(54) BIO-ADHESIVE MATERIALS WITH CATIONIC SURFACE PEPTIDE

(71) Applicant: MIYOSHI AMERICA, INC., Dayville, CT (US)

(72) Inventors: Shoji TAKEKAWA, Shrewsbury, MA (US); Ryo Ohara, Tokyo (JP)

(21) Appl. No.: 19/090,752

(22) Filed: Mar. 26, 2025

(51) Int. Cl. A61K 8/64; A61Q 19/00

(52) U.S. Cl. CPC A61K 8/64; A61Q 19/00; A61K 2800/10; A61K 2800/622; A61K 2800/651

(57) ABSTRACT

The present disclosure relates generally to bio-adhesive materials having a surface containing one or more cationic surface peptides, formulations containing the materials, and methods of making a material comprising one or more cationic surface peptides on the surface thereof comprising contacting the substrate material with at least one cationic surface peptide to immobilize the at least one cationic surface peptide to the surface of the substrate material. The materials have superior bio-adhesion to biological substrates, reduced aggregation, can be used in formulations without significant aggregation of the materials, and can be used without the need to add a hydrophobic surface treatment, or a film-forming ingredient.

### FIELD

The present disclosure relates generally to bio-adhesive materials having a surface containing one or more cationic surface peptides, formulations containing the materials, and methods of making a material comprising one or more cationic peptides on the surface thereof comprising contacting the material with at least one cationic peptide. The materials have superior bio-adhesion to biological substrates, reduced aggregation, can be used in formulations without significant aggregation of the materials, and without the need to add a hydrophobic surface treatment, or a film-forming ingredient.

### BACKGROUND

The information provided below is not admitted as prior art to the embodiments, but is provided solely to assist in a more complete understanding thereof.

A significant amount of various powders are conventionally used for making makeup, skincare products, sunscreens, toiletries, and other products marketed and distributed by the personal care industry. Powders dispersed in various product forms such as water base solution, water gel, water-in-oil (W/O) and oil in water (O/W) emulsion formulas, may suffer from poor dispersibility, and inferior product stability, which can result in the formation of aggregates, agglomerates and flocculation. These results can be due to the nature of powder's physical properties, including particle size, surface activity, charge, polarity and specific gravity, to name a few.

Untreated powder agglomerates easily due to several surface properties (including surface charge, surface polarity etc.). In order to solve this problem and to thereby improve dispersibility and stability of powders, surface treatments with various treating agents have been proposed, as well as the use of film-forming ingredients. Agents and methods for surface treating powders vary depending on the aim of the treatment. A treating agent may be selected in view of properties of the surface to be treated and its interaction with a dispersion medium. Known methods include, for instance, lipophilization with oils or metal soaps, hydrophilization treatment with surfactants or silica, and hydrophobization with silicone oils.

In recent years, powders have been developed to provide long lasting cosmetics with a smoother consistency. In obtaining these desirable traits, the focus has largely been on the hydrophobic properties of the surface treatments on powders and pigments, and improvements in the dispersibility of surface treated powders into an oil phase. However, when powders are used in cosmetic systems, such as foundations, lip sticks, lotions, or creams, the powders typically have to be dispersed in an aqueous phase, due to the hydrophilic nature of most cosmetic powders. To disperse non-hydrophobic powders in an aqueous phase, multiple emulsifiers are often used. Without these emulsifiers, dispersions in water-based systems often become problematic. The use of emulsifiers can be disadvantageous, however, with respect to producing a sticky, heavy feeling to the composition.

It also is known that direct contact of inorganic and organic cosmetic powders with the skin may lead to the absorption of water on the skin surface, thus altering the natural hydrophilic and lipophilic balance, which may cause localized dehydration effects and consequently an unpleasant feeling by those using these products. In addition, the lack of homogeneity of the powders used, having different physical features from one another, may ultimately generate clearly perceptible defects. Cosmetic powders therefore are typically treated to modify the surface of the powder to provide improved dispersibility, homogeneity and stability and to reduce the deleterious effects caused by direct contact with the skin.

There are proposed a variety of surface-treating methods. In one method, a silicone oil (for instance, methyl polysiloxane, methyl hydrogen polysiloxane or alkyl silane with the number of carbon atoms of an alkyl portion being not more than 10) is dissolved into a solvent as a surface-treating agent, which then is added and mixed into a powder, and the surface treatment is baked onto the powder by heating after the drying process. In another method, while a powder and octyl triethoxy silane or the like are being dispersed into an organic solvent by using a media grinder, the surface of the powder is treated with an organic silicon compound such as octyl triethoxy silane (JP-A 08-104606). Another method involves stirring and mixing with a Henschel mixer N-octyl trimethoxy silane or N-octyl triethoxy silane as an alkyl silane compound, and a reaction is completed with the powder under heating, and the resultant treated powder is pulverized by a hammer mill (JP-A 2001-181136). In another method, a silicone compound such as methyl hydrogen polysiloxane or the like is emulsified by dispersing it in water, and surfaces of powder particles are coated by mixing the emulsion to the powder (JP-A 09-268271).

JP-B 06-59397 discloses a jet method in which after a metal soap, an organic silicon compound in which a reactive group such as a hydrogen group or the like is bonded to a silicon atom, and a powder are mixed, the mixture is pulverized by a miller using an ejecting stream simultaneously with the surface treatment. JP-A 2002-80748 discloses a method in which in order to improve dispersability of a powder, coating is effected with surface treating agents for an A layer and a layer B by a jet method. Another method involves mixing a silica compound in water, ethanol and aqueous ammonia, and therein dispersing titania powder to prepare a pre-mix 1. Separately, tetraethoxysilane, water and ethanol were mixed to prepare pre-mix 2. Pre-mix 2 was added to pre-mix 1 under stirring with a magnetic stirrer, at a constant rate over 2 hours. The mixture obtained was aged for 12 hours. The coating formation and aging were performed at 25° C. Thereafter, the solution was filtered by suction and the filtrate was dried with hot air at 50° C. for 12 hours to obtain silica-coated powder. This process is disclosed in U.S. Pat. No. 6,534,044, the disclosure of which is incorporated by reference herein in its entirety.

U.S. Pat. No. 5,496,544, the disclosure of which is incorporated by reference herein in its entirety, discloses a skin cosmetic composition consisting of an anhydrous powder comprising a solid powder phase mixed with a fat-based binder which contains a silicone mixture comprising at least one silicone oil, at least one silicone wax, at least one silicone resin, and optionally at least on silicone rubber and optionally at least one phenyl dimethicone. However, in U.S. Pat. No. 5,496,544, the anhydrous powder undergoes a physical treatment by the fat-based binder. Therefore, in the cosmetic composition from U.S. Pat. No. 5,496,544, the absence of a covalent chemical bond between the powder phase and fat-based binder has the drawback of an easy extraction of the latter from the powder phase. Also, in the cosmetic composition from U.S. Pat. No. 5,496,544, the powder phase coating consists of complex mixtures of silicones which confer a different kind of sensorial effects on the skin itself.

EP 1 116 753 describes a powder treated with reactive silicone comprising a powder surface-coated with a silicone compound, in which the amount of hydrogen generated from Si—H groups left on the surface of the silicone-treated powder is not greater than 0.2 ml/g of the treated powder and a contact angle between the water and the treated powder is at least 100°. However, the direct reaction between methyl hydrogen polysiloxane containing reactive Si—H bonds and the powder surface described in EP 1 116 753 fails to reach completion and it has the disadvantage to release some H₂ over time, which is the cause of several drawbacks for the obtained cosmetic powder. Indeed, on the one hand the generation of H₂ may cause the containers carrying the powder to swell and deteriorate, on the other hand the powder itself may harden and break.

A number of different surface treatment agents have been used to impart hydrophobicity to the cosmetic powders, both organic and inorganic, and are disclosed in, for example, U.S. Pat. Nos. 4,622,074; 4,863,800; 5,368,639; 5,458,681; 5,744,126; 6,156,324; 6,200,580; 6,251,411; 6,296,860; 6,416,573; 6,482,441; 6,790,452; 7,276,113; 7,374,783; 8,105,691; 8,591,925; 9,011,893; 9,114,266; 9,597,268; 9,597,269; and 10,196,524, and U.S. Patent Application Publication Nos. 2004/0247542; 2005/0147630; 2008/0118538; 2011/0110995; 2011/0250250; 2011/0318286; 2012/0156268; 2012/0308628; 2014/0219941; 2014/0363387; 2017/0281512; 2022/0127157; 2022/0127158; 2022/0249333; 2022/0265524; and 2022/0273549, the disclosures of which are incorporated by reference herein in their entireties.

It is desirable to formulate compositions containing high concentration powder dispersions that have improved flowability, as well as re-dispersibility. It also would be desirable to impart surface characteristics to materials without the need for surface treatment agents, or film-forming ingredients used to impart hydrophobicity to the powders. It further would be desirable to provide materials having improved bio-adhesion to biological substrates such as nails, chitin, skin, hair, eyebrows, eyelashes, etc., without significant aggregation of the material. Other objects and advantages will become apparent from the following disclosure. While certain drawbacks and disadvantages have been described with respect to the state of the art, the embodiments described herein are not to be construed to exclude some or all of the features described above. Indeed, aspects of the embodiments may include features known in the art, without suffering from their previously known adverse effects.

Other objects and advantages will become apparent from the following disclosure. While certain drawbacks and disadvantages have been described with respect to the state of the art, the embodiments described herein are not to be construed to exclude some or all of the features described above. Indeed, aspects of the embodiments may include features known in the art, without suffering from their previously known adverse effects.

### SUMMARY

The embodiments described herein relate to bio-adhesive compositions in which the substrate material can be a cosmetic agent (e.g., silica bead, mineral, pigment, substrate, etc.), or a consumer product (coloring agent, flavor, fragrance, etc.). The bio-adhesive composition may include a powder or other material (e.g., substrate) having one or more cationic peptides on its surface. Some embodiments include a method of making a material having one or more cationic peptides on the surface thereof that includes adding a solution containing at least one cationic peptide to a material with agitation to disperse the material and cationic peptide solution, adding one or more multi-valent metal-containing salt to the dispersed solution, and mixing to prepare a bio-adhesive powder having one or more cationic peptides chemically bound to, or immobilized on the surface of the material. The method may include separating the bio-adhesive powder from the mixture or dispersion, including by precipitation and or drying to form bio-adhesive powder particles having at least one cationic peptide immobilized on the surfaces thereof.

According to other embodiments, there is provided a formulation that includes: (a) at least one bioadhesive composition comprising one or more substrate materials having one or more cationic peptides bound to or immobilized on the surfaces thereof; and (b) an acceptable carrier. According to other embodiments, there is provided a cosmetic formulation that includes at least one bioadhesive cosmetic powder having one or more cationic peptides bound to or immobilized on the surface thereof, in which the cosmetic formulation contains a high concentration of the at least one bioadhesive cosmetic powder. In these and other embodiments, the at least one substrate material having one or more cationic peptides bound to or immobilized to the surface thereof does not contain a hydrophobic surface treatment agent or film-forming ingredient.

Still other aspects and advantages of the embodiments will become readily apparent to those having ordinary skill in the art from the following detailed description, wherein particularly preferred embodiments are shown and described, simply by way of illustration. As will be realized the preferred embodiments include other and different embodiments, and its several details are capable of modifications in various obvious respects. Accordingly, the description is to be regarded as illustrative in nature and not as restrictive.

### BRIEF DESCRIPTION OF THE DRAWINGS

The embodiments may be better understood from the following detailed description when read in connection with the accompanying drawings.

FIG. 1 is an electron microscopic picture of hair contacted with silica beads surface treated with poly-lysine.

FIG. 2 is an electron microscopic picture of hair contacted with iron oxide pigments surface treated with poly-lysine.

FIG. 3 is an electron microscopic picture of hair contacted with un-treated silica beads.

### DETAILED DESCRIPTION OF A PREFERRED EMBODIMENT

The following definitions and non-limiting guidelines are provided to assist in better understanding the detailed description of herein. The headings (such as “Background” and “Summary,”) and sub-headings used herein are intended only for general organization of topics within the disclosure of the embodiments, and are not intended to be limiting. For example, subject matter disclosed in the “Background” may include aspects of technology within the scope of the embodiments, and may not constitute a recitation of prior art. Subject matter disclosed in the “Summary” is not an exhaustive or complete disclosure of the entire scope of the embodiments. Classification or discussion of a material within a section of the specification as having a particular utility (e.g., as being an “active” or a “carrier” ingredient) is made for convenience, and no inference should be drawn that the material must necessarily or solely function in accordance with its classification herein when it is used in any given composition.

The citation of references herein does not constitute an admission that those references are prior art or have any relevance to the patentability of the embodiments disclosed herein. Any discussion of the content of references cited in the Background is intended merely to provide a general summary of assertions made by the authors of the references, and does not constitute an admission as to the accuracy of the content of such references.

The description and specific examples, while indicating embodiments, are intended for purposes of illustration only and are not intended to be limiting. Moreover, recitation of multiple embodiments having stated features is not intended to exclude other embodiments having additional features, or other embodiments incorporating different combinations the stated of features. Examples are provided for illustrative purposes of how to make and use the compositions and methods described herein, unless explicitly stated otherwise, are not intended to be a representation that given embodiments have, or have not, been made or tested.

As used herein, the words “preferred” and “preferably” refer to embodiments that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope thereof. In addition, the compositions and the methods may comprise, consist essentially of, or consist of the elements described therein.

As used throughout, ranges are used as a short-hand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls.

Throughout this description, the use of the term “about” or “approximately” is intended to denote an approximation of the number, which includes the number modified by the term, and a reasonable deviation from that term, including standard measurement errors. Unless otherwise specified, all percentages and amounts expressed herein and elsewhere in the specification should be understood to refer to percentages by weight. The amounts provided are based on the active weight of the material. The recitation of a specific value herein is intended to denote that value, plus or minus a degree of variability to account for errors in measurements. For example, an amount of 10% or about 10% may include 9.5% or 10.5%, given the degree of error in measurement that will be appreciated and understood by those having ordinary skill in the art.

As used herein, the term “cosmetic composition” means a composition that is intended to be applied onto the consumer's skin, particularly onto the facial skin or onto the body skin area or onto hair, so as to regulate the condition of the skin and/or to improve the appearance of the skin and hair. The term “powder” denotes any material having a particle size within the range of from about 0.01 μm to 1,000 μm used for cosmetics. The term “average primary particle size” of powder treated with polysaccharide denotes the equivalent volume mean primary particle size of the elementary powder treated with polysaccharide. The average primary particle size is measured on the powder treated with polysaccharide, before being treated. The expression “ultra-fine powder” denotes a powder having an average particle size of less than about 100 μm, but not less than 0.1 μm (i.e., greater than 100 nm, but less than 100 μm).

Throughout this description, the term “foundation” means a cosmetic composition that is intended to be applied onto the consumer's skin, particularly, onto the facial skin, body skin and hair so as to provide coverage and/or to mask skin irregularities and/or skin imperfections and/or skin tonal variations. The term “chalkiness” means the white hue which is observed onto skin after applying onto skin, particularly darker skin. The term “pastiness” means the white hue that may be observed on the skin after applying onto skin, particularly lighter skin.

All percentages, ratios and proportions herein are by weight, unless otherwise specified. All such weights as they pertain to listed ingredients are based on the active level, unless otherwise specified.

Embodiments described herein include bio-adhesive compositions in which the substrate material can be a cosmetic agent (e.g., silica bead, mineral, pigment, substrate, etc.), or a consumer product (coloring agent, flavor, fragrance, etc.). The bio-adhesive composition may include a powder or other material (e.g., substrate) having one or more cationic peptides on its surface.

The substrate material can be a cosmetic agent. Suitable cosmetic agents include cosmetic powders selected from substrates, pigments, and extenders. Substrates and pigments typically comprise or consist of a material compatible or acceptable for cosmetic and makeup products, personal care products, sunscreens, and pharmaceutical products. Substrates and pigments are typically in the form of a powder, which is a solid, dry material consisting of small, flowable particles. Particular classes of powder materials are inorganic and organic particles, beads, crystals, clays, metals, metal oxide powders, plastics and fillers for plastic suitable for cosmetic use. The substrate material also can be a substrate material used in consumer products, such as a coloring agent, flavorant, fragrance, and the like.

The at least cationic surface peptide can optionally be chemically immobilized or adsorbed onto the surface substrate material. Chemical linkage or immobilization of the cationic surface peptide to a substrate material differs from adsorption in that surface treated material has a more uniformly chemically bound reaction product. Chemical linkage or immobilization tends to reduce movement and/or rearrangement of any material linked or attached onto the surface of the modified substrate material. For example, a cationic surface peptide that is linked or attached to the surface of a cosmetic powder will have less mobility than a cationic surface peptide that is attached or linked to the surface of a powder by virtue of adsorption.

In order to facilitate or enhance immobilization of cationic surface peptides to the substrate material, a reaction may be created by a water-soluble compound having a lipophilic or hydrophilic moiety being absorbed onto the surface of the substrate material. As a non-limiting example, addition of a water-soluble salt of a polyvalent metal, such as magnesium, calcium, aluminum, titanium, zinc or a zirconium salt (e.g., zirconium sulfate or chloride), or an alkaline salt, such as a sodium, potassium, lithium, ammonium, or an amine salt, can produce a chemical linkage. These metals typically are present in the form of a salt, such as a sulfate salt (e.g., aluminum sulfate, and the like). The reaction provides a cationic surface peptide chemically immobilized onto the surface of the substrate material particles. In contrast, conventional coating a substrate with a cationic surface peptide involves absorbing the cationic surface peptide onto the surface of the substrate.

During treatment with one or more cationic surface peptides, the surface of one or more substrate materials becomes modified. Including an oil, such as a cosmetically acceptable oil (a single oil or mixture of oils) during a treatment in which the substrate surface is modified invites oil at the same time as the particles become attached or linked to each other. Cationic surface peptides and oil in combination may function as a “glue” to attach or link particles, and other components optionally present, to each other. A mixture of two or more different substrate materials during such surface treatment with the one or more cationic surface peptides may result in forming composites, which are typically randomly and uniformly distributed onto the surface. Thus, oils, emulsifiers, etc., can be present in a mixture with one or more substrates and pigments when contacted with one or more cationic surface peptides.

When the substrate material is at least one cosmetic powder, the at least one cosmetic powder material typically is a mineral photoprotective agent in the form of a pigment, such as, for example, metal oxides such as, for example, ultra-fine powders of titanium oxide (amorphous or crystallized in rutile and/or anatase form), of iron oxide, of zinc oxide, of aluminum hydroxide, of zirconium oxide, of cerium oxide, or mixtures thereof. The one or more ultra-fine powders typically have an average particle size of from about 0.1-100 μm in diameter, or from about 0.2 to about 80 μm, or from about 0.5 to about 50 μm, or from about 1 to about 35 μm.

Non-limiting examples of inorganic pigments include white titanium dioxide pigments (e.g., rutile, anatase, and ultrafine TiO₂), zinc oxides (e.g., ultrafine ZnO), or aluminum hydroxide. Other inorganic pigments include zirconium oxide, zirconium dioxides, iron oxides (including yellow, red, brown, green and black iron oxides), ultramarines (such as ultramarine blue, ultramarine violet, ultramarine pink, etc.), pearl pigments (e.g., mica, titanated mica, bismuth oxychloride, etc.), manganese violet, Prussian blue, chromium oxides, chromium hydroxides, and carbon black. Non-limiting examples of organic pigments include “lake” dyes, β-carotene, carmine, chlorophyll and the like.

The substate material also may be an extender pigment such as, but not limited to Excel Mica, Excel Pearl and Powder La Vie sold by Miyoshi Kasei, Inc.; white pigments such as titanium dioxide, zinc oxide and cerium oxide; color pigments such as red iron oxide, yellow iron oxide, black iron oxide, chromium oxide, chromium hydroxide, Prussian blue, ultramarine, inorganic blue pigment, carbon black, titanium oxide, mango violet, cobalt violet, laked tar dye and laked natural dye; bright pigments such as bismuth oxychloride, mica titanium, fish scale guanine, a powder obtained by coating synthetic mica with titanium dioxide, a powder obtained by coating silica flakes with titanium dioxide as sold under a trade name METASHINE® by Nippon Sheet Glass Co., Ltd., a powder obtained by coating alumina flakes with tin oxide and titanium dioxide, a powder obtained by coating aluminum flakes with titanium dioxide, a powder obtained by coating copper flakes with silica as sold by Eckart, U.S.A., or a powder obtained by coating bronze flakes with silica and a powder obtained by coating aluminum flakes with silica.

Other suitable extender pigments may include one or more of mica, sericite, talc, kaolin, synthetic mica, muscovite, phlogopite, epidolite, biotite, calcium carbonate, magnesium carbonate, calcium phosphate, alumina, magnesium oxide, aluminum hydroxide, barium sulfate, magnesium sulfate, silicic acid, silicic anhydride, magnesium silicate, aluminum silicate, aluminum magnesium silicate, calcium silicate, barium silicate, strontium silicate, silicon carbide, magnesium aluminate, magnesium metasilicate aluminate, chlorohydroxyaluminum, clay, bentonite, zeolite, smectite, hydroxyapatite, ceramic powder, boron nitride and silica.

The substrate material also can comprise an organic powder, non-limiting examples of which include a wool powder, a polyamide powder, a polyester powder, a polyethylene powder, a polypropylene powder, a polystyrene powder, a polyurethane powder, a benzoguanamine powder, a tetrafluoroethylene powder, a polymethyl methacrylate powder, a cellulose powder, a silk powder, a silicone powder, a silicone rubber powder, a styrene acrylic copolymer, a divinylbenzene·styrene copolymer, synthetic resin powders such as a vinyl resin, a urea resin, a phenol resin, a fluoro resin, a silicon resin, an acrylic resin, a melamine resin, an epoxy resin and a polycarbonate resin, a fine crystalline fibrous powder, a starch powder, an acylated lysine powder, a long-chain alkyl phosphate metallic salt powder, or a metal soap powder. Other substrate materials include, for example, beads such as silica and mica beads.

Suitable powder materials include inorganic pigments such as, but not limited to titanium dioxides, zinc oxides, zirconium dioxides, iron oxides (including yellow, red, and black), ultramarines (such as ultramarine blue, ultramarine violet, etc.), and manganese violet. The powder material may be a mixture of any or all of the suitable powder materials.

The at least one cationic surface peptide preferably is a peptide of a repeating unit of a cationic amino acid, including but not limited to arginine, lysine, and histidine. The peptide may contain 3 or more repeating units of the cationic amino acid, and mixtures thereof. The cationic surface peptide may contain from 4-50 amino acids, or from 20-45, or 25-35, or any value therebetween. In one embodiment, the cationic surface peptide is selected from poly-L-lysine, poly-D-lysine, poly-L-arginine and poly-D-arginine. A particularly preferred cationic surface peptide is poly-lysine containing at least one positively charged amino group having the formula below:

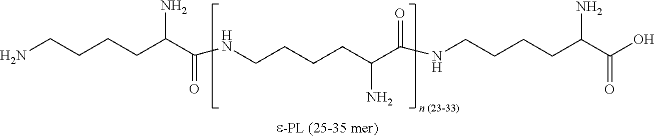

The substrate material whose surface has been modified with at least one cationic surface peptide usually will have an average treatment ratio of from about 0.05 to about 20% by weight of cationic surface peptide based on the weight of the substrate material (or from about 0.05 to about 20 parts of cationic surface peptide by weight per 100 parts by weight of substrate material), or from about 0.1 to about 10% by weight of cationic surface peptide.

The substrate material also may be treated with from about 0.1 to about 10% cationic surface peptide, by weight of substrate material, or from about 2 to about 6%, or from about 3 to about 5% cationic surface peptide, by weight of substrate material. The amount of cationic surface peptide also may vary depending on the type of substrate material. For example, for ultrafine powder, such as silica having a larger surface area, more cationic surface peptide may be used, e.g., double or triple the amount used for powder having smaller surface area. Using the guidelines provided herein, persons having ordinary skill in the art will be capable of determining an appropriate amount of cationic surface peptide to use, depending on the type of substrate material being treated.

Another embodiment disclosed herein includes a method of making a bio-adhesive material comprising a substrate material comprising one or more cationic peptides on the surface thereof. In accordance with one method, the method includes preparing a solution containing one or more substrate materials in which the solution may contain one or more basic materials such as sodium hydroxide. The solution may be mixed at high speed, e.g., from 10-10,000 rpm, to provide a homogeneous solution and/or dispersion containing the one or more substrate materials. The substrate material can be added to the solution at a temperature of from about 25 to about 75° C., or from about 40 to about 60° C., or at about 50° C., and mixed in a disperser for a period of time sufficient to homogenize the mixture. Any dispersing and/or mixing apparatus can be used. A suitable disperser may include a ROBOMIX® disperser, commercially available from Primix Corporation, Osaka, Japan. The mixture can be mixed for anywhere from about 10 minutes to an hour, or from about 15 minutes to 45 minutes, or from about 18 minutes to 30 minutes, or for about 20 minutes, until the mixture is adequately homogenized.

One or more cationic surface peptides (optionally in the form of a solution) then can be added to the homogeneous solution and/or dispersion and again mixed at high speed, e.g., from 10-10,000 rpm, to form a homogenous dispersion of the one or more substrate materials and the one or more cationic surface peptides. The mixture of the substrate solution and the cationic peptide solution can be mixed for a period of time within the range of from about 5 to about 60 minutes, or from about 10 to about 40 minutes, or from about 15 to about 25 minutes, until adequately dispersed to thereby form a homogeneous dispersion. In an embodiment, the one or more cationic surface peptides are present, preferably as an aqueous solution, in the homogeneous dispersion in an amount of from about 0.1 to about 75% by weight, or from about 0.2 to about 65% by weight, or from about 0.6 to about 60% by weight, or from about 1.0 to about 50% by weight, based on the total weight of homogeneous dispersion.

A solution containing a multivalent metal ion-containing salt (e.g., aluminum sulfate, aluminum chloride, calcium chloride, magnesium chloride, etc.) then can be added to the homogenous dispersion of the one or more substrate materials and the one or more cationic surface peptides in an amount to neutralize the dispersion and also to immobilize the one or more cationic surface peptides on the surface of the one or more substrate materials to form a surface treated substrate material. The multivalent metal ion-containing salt (e.g., neutralizing agent) may be added to the homogenous dispersion to bring the pH of the dispersion to a value within the range of from about 2 to about 10, or from about 3 to about 8, or from about 4 to about 7, or about 4.0. Any neutralizing agent can be used in the embodiments, and a suitable neutralizing agent is aluminum sulfate. The neutralizing agent can be metered into the mixture until the pH reaches the desired value. Once the final pH is reached, the product then can be recovered from the mixture using any suitable mechanism, including filtration, and then drying. The substrate material treated with the one or more cationic surface peptides can be dried at a temperature of between about 75 to about 200° C., or from about 90 to about 150° C., or at about 105° C., for a period of time sufficient to dry the powder. The substrate material treated with the one or more cationic surface peptides may be subjected to drying for a period of from about 5 to about 35 hours, or from about 10 to about 20 hours, or from about 15 to about 17 hours, or about 16 hours, to produce, in the embodiments disclosed above.

In an embodiment, the multivalent metal ion-containing salt is present in the solution in an amount of from about 1% to about 65%, or from about 3% to about 50%, or from about 5% to about 30%, by weight, or any value therebetween. In one embodiment, the multivalent metal ion-containing salt is Al₂(SO₄)₃, and the solution is added slowly to the homogenous dispersion of the one or more substrate materials and the one or more cationic surface peptides until the desired pH is obtained. In one embodiment, the weight ratio of the cationic surface peptide(s) and the multivalent metal ion-containing salt is from about 0.01 to about 10.0, or from about 0.02 to about 7.0, or from about 0.05 to about 2.0, or any value therebetween.

The surface treated substrate material then may be separated from the dispersion using conventional separation techniques including, but not limited to, filtration, drying, neutralization and precipitation, and the like. In one embodiment, the solid surface treated substrate material is separated by filtration, and then dried as described previously.

The surface treated substrate material may be combined with additional components to form a composition suitable for its end use. When used in a cosmetic formulation, the surface treated substrate material may be admixed with one or more surfactants. Suitable surfactants include partially crosslinked polyether-modified silicone, a partially crosslinked polyglycerin-modified silicone, or a combination thereof, including those disclosed in U.S. Pat. No. 8,409,551, the disclosure of which is incorporated by reference herein in its entirety. Partially crosslinked polyether-modified silicones are three-dimensional crosslinked materials in which organopolysiloxane chains have been crosslinked via polyether chains. Specific examples of these partially crosslinked polyether-modified silicones include materials referred to using the names (dimethicone/(PEG-10/15)) crosspolymer, (PEG-15/lauryl dimethicone) crosspolymer and (PEG-15/lauryl polydimethylsiloxyethyl dimethicone) crosspolymer. These materials are available commercially in a form of being swollen with and containing a silicone or other oils, and are marketed under product names such as KSG-210, 240, 310, 340 and 320Z (all manufactured by Shin-Etsu Chemical Co., Ltd.).

Partially crosslinked polyglycerin-modified silicones are three-dimensional crosslinked materials in which organopolysiloxane chains have been crosslinked via polyglycerin chains. Specific examples of these partially crosslinked polyglycerin-modified silicones include materials referred to using the names (dimethicone/polyglycerin-3) crosspolymer, (lauryl dimethicone/polyglycerin-3) crosspolymer, and (polyglyceryl-3/lauryl polydimethylsiloxyethyl dimethicone) crosspolymer. These materials are available commercially as gels containing silicone oils and other oils, and are marketed under product names such as KSG-710, 810 and 820Z (all manufactured by Shin-Etsu Chemical Co., Ltd.). It is preferred to use as the surfactant a polyglycerin-modified silicone, and particularly preferred to use lauryl polyglyceryl-3 polydimeethylsiloxyethyl dimethicone, commercially available from Shin-Etsu Chemical Co., Ltd. as KF6105.

Suitable silicone oils include, for example, linear and cyclic siloxanes. In one embodiment, a cyclic siloxane is used, which may be any one or more of dihydrogenhexamethyl cyclotetrasiloxane, trihydrogen pentamethyl cyclotetrasiloxane, tetrahydrogen tetramethyl cyclotetrasiloxane, dihydrogen octamethyl cyclopentasiloxane, trihydrogen heptamethyl cyclopentasiloxane, tetrahydrogen hexamethyl cyclopentasiloxane, and pentahydrogen pentamethyl cyclopentasiloxane. A particularly preferred silicone oil is a cyclopentasiloxane, commercially available from Shin-Etsu Chemical Co., Ltd. as KF-995.

If used as a cosmetic dispersion, the cosmetic dispersions may contain the at least one surfactant in an amount of from about 0.01% to about 25% by weight, based on the weight of the dispersion. Alternatively, the cosmetic dispersions may contain the at least one surfactant in an amount of from about 0.01% to about 20% by weight, or from about 0.01% to about 15% by weight, or from about 0.01% to about 10% by weight, based on the weight of the dispersion.

The cosmetic dispersions may contain the at least one cosmetic powder having one or more cationic peptides on the surface thereof in an amount of from about 45% to about 95% by weight, based on the weight of the dispersion. Alternatively, the cosmetic dispersions may contain the at least one cosmetic powder having one or more cationic peptides on the surface thereof in an amount of from about 50% to about 90% by weight, or from about 55% to about 85% by weight, or from about 60% to about 80% by weight, based on the weight of the dispersion.

The cosmetic dispersions may contain the at least one silicone oil in an amount of from about 1% to about 50% by weight, based on the weight of the dispersion. Alternatively, the cosmetic dispersions may contain the at least one silicone oil in an amount of from about 5% to about 45% by weight, or from about 7% to about 42% by weight, or from about 10% to about 40% by weight, based on the weight of the dispersion.

The substrate material containing one or more cationic surface peptides on the surface thereof may be combined with other conventional components useful in various cosmetic compositions. Any cosmetically acceptable vehicle may be used together with the surface treated substrate material. Such vehicles may include, for example, water, glycerin, dimethicone, beeswax, glyceryl stearate, and the like. Other ingredients normally used in cosmetics also may be present, when desired. For example, inorganic powders such as talc, kaolin, sericite, muscovite, phlogopite, red mica, biotite, synthetic mica, lithia mica, vermiculite, magnesium carbonate, calcium carbonate, diatomite, magnesium silicate, calcium silicate, aluminum silicate, barium silicate, barium sulfate, strontium silicate, wolframic acid metal salt, or silica, hydroxyapatite, zeolite, boron nitride, ceramic powder, organic powders such as nylon powder, polyethylene powder, polystyrene powder, benzoguanamine powder, polyfluoridation ethylene powder, di-styrene benzene polymer powder, epoxy powder, acrylic powder, silicone powder, microcrystalline cellulose, inorganic white pigments such as titanium dioxide and zinc oxide, inorganic red system pigments such as iron oxide (red iron oxide) and titanic acid irons, inorganic brown system pigments such as γ-iron oxides, inorganic yellow system pigments such as yellow soil and yellow iron oxides, inorganic black color system pigments such as tetravalent acid iron oxide, carbon black, inorganic violet system pigments such as mango violet, cobalt violet, inorganic green system pigments such as chromium oxide, chromium hydroxide, and titanic acid cobalt, inorganic blue system pigments such as ultramarine blue, and prussian blue, pearl pigments such as titanium dioxide covered mica, titanium dioxide covered bismuth oxychloride, bismuth oxychloride, titanium dioxide covered talc, fish scale foil, colored titanium dioxide covered mica, metal powder pigment such as aluminum powder, copper powder, colored composite pigments such as iron-doped zinc oxide and iron-doped titanium dioxide.

Other pigments may be used, such as red No. 201, red No. 202, red No. 204, red No. 205, red No. 220, red No. 226, red No. 228, red No. 405, orange-colored No. 203, orange-colored No. 204, yellow No. 205, yellow No. 401 and blue No. 404, organic chlorophyll pigment such as FD&C Red No. 3, red No. 104, red No. 106, red No. 227, red No. 230, red No. 401, red No. 505, orange-colored No. 205, FD&C Yellow No. 4, yellow No. 5, yellow No. 202, yellow No. 203, orange-colored No. 3 and zirconium, barium, or aluminum lake of blue No. 1, natural colorants such as β-carotene, hydrocarbon oils such as squalane, mineral oil, petroleum jelly, micro crystalline wax, ozokerite, ceresin, myristic acid, palmitic acid, stearic acid, oleic acid, isostearic acid, cetyl alcohol, hexadecyl alcohol, oleyl alcohol, cetyl 2-ethylhexanoate, 2-ethylhexyl palmitate, 2-octyldodecyl myristate, neo-pentylglycol di-2-ethylhexanoate, glyceryl tri-2-ethylhexanoate, 2-octyldocyl oleate, isopropyl myristate, glyceryl triisostearate, caprylic/capric triglyceride, olive oil, avocado oil, yellow bees wax, myristyl myristate, mink oil, lanolin oil, silicone oil, higher fatty acid oil, ester oils of fatty acids, higher alcohol, oil components of wax groups, cyclopentasiloxanes, dimethicones, trimethylsiloxysilicates, and organic solvents such as acetone, toluene, butyl acetate, and ester acetate can be used in various amounts.

Resins such as alkyd resin, urea-formaldehyde resin, Nylon-12, plasticizers such as camphor, acetyl tributyl citric acid, ultraviolet absorbing agents, antioxidants, antiseptics, emulsifiers, surfactants, stabilizers, defoamers, moisturizing agents, perfumes, water, alcohol, and thickeners can also be used. Non-limiting examples of emulsifiers include cetyl dimethicone copolyol, polygyceryl-4 isosteatrate, glyceryl stearate, PEG-100 stearate, cetyl alcohol, dicetyl phosphate, and ceteth-10 phosphate isostearic acid.

Surfactants typically include nonionic forms. Non-limiting examples of nonionic surfactants include polyoxyalkylene (PEG or/and PPG) type nonionic emulsifiers having structures:

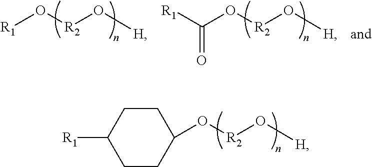

wherein R₁ is selected from the group consisting of alkyl, alkylamide, alkenyl, alkynyl, alkoxy, aryl, cycloalkyl, and arylalkyl group, each of which may be substituted by one or more hydroxy group, and may further be substituted by one or more alkoxyl, carboxyl, or oxo group. R₁ has a carbon number of C₈ to about C₂₄; R₂ is selected from the group consisting of —C₂H₄—, —C₃H₆—, and —C₄H₈—.

The substrate material treated with the one or more cationic peptides can be used in a cosmetic composition that contains conventional cosmetic additives. For example, the composition may include up to about 25 wt % of a non-volatile oil. The non-volatile oil may be comprised of an organic, UV-active material that functions as a UV-protective agent (a “sun block”). Preferably, two or more organic, UV-actives are used to provide a wide spectrum of protection in the UV region. For example, a combination of at least one UV protecting agent that mainly provides protection against UVA light, and at least one UV protecting agent that mainly provides protection against UVB light, may be used.

A wide variety of conventional UV protecting agents are suitable for use herein. Non-limiting exemplary organic, UV-actives include: 2-ethylhexyl-p-methoxycinnamate (commercially available as PARSOL MCX), butylmethoxydibenzoyl-methane, 2-hydroxy-4-methoxybenzo-phenone, 2-phenylbenzimidazole-5-sulfonic acid, octyldimethyl-p-aminobenzoic acid, octocrylene, 2-ethylhexyl N,N-dimethyl-p-aminobenzoate, p-aminobenzoic acid, 2-phenylbenzimidazole-5-sulfonic acid, octocrylene (Parsol 340, DSM), oxybenzone, homomenthyl salicylate, octyl salicylate, 4,4′-methoxy-t-butyldibenzoylmethane, 4-isopropyl dibenzoylmethane, 3-benzylidene camphor, 3-(4-methylbenzylidene) camphor, Eusolex™ 6300, avobenzone (Parsol 1789, DSM), avobenzone, PABA, octyldimethyl-PABA, Phenylbenzimidazole sulfonic acid, Cinoxate, Dioxybenzone (Benzophenone-8), Oxybenzone (Benzophenone-3), Homosalate, Menthyl anthranilate, Octisalate, Sulisobenzone, Trolamine salicylate, Terephthalylidene Dicamphor Sulfonic Acid, 4-Methylbenzylidene camphor, Methylene Bis-Benzotriazolyl Tetramethylbutylphenol, Bis-ethylhexyloxyphenol methoxyphenol triazine, bisimidazylate, Drometrizole Trisiloxane, Octyl triazone, Diethylamino Hydroxybenzoyl Hexyl Benzoate, Iscotrizinol, Polysilicone-15, Amiloxate, Ethylhexyl Dimethoxybenzylidene Dioxoimidazolidine Propionate, and mixtures thereof.

In addition to a UV-active, the non-volatile oil may comprise an ancillary oil which may be a solvent for one or more of the UV-active oils. The ancillary oil may provide desirable cosmetic properties such as emolliency and a good “skin feel.” A preferred, but non-limiting ancillary oil is isopropyl myristate.

Non-volatile cosmetic emollient oils having a relatively high boiling point and function as a skin feel modifiers include, but are not hydrocarbons, fatty alcohols, fatty acids, non-volatile silicone oils, and esters such as glycerides and glycol esters.

Suitable ancillary oils include, but are not limited to isotridecyl isononanoate, isostearyl isostearate, isocetyl isosteatrate, isopropyl isostearate, isodecyl isonoanoate, cetyl octanoate, isononyl isononanoate, isocetyl myristate, isotridecyl myristate, isopropyl myristate, isostearyl palmitate, isocetyl palmitate, isodecyl palmitate, isopropyl palmitate, octyl palmitate, caprylic/capric acid triglyceride, glyceryl tri-2-ethylhexanoate, neopentyl glycol di(2-ethyl hexanoate), diisopropyl dimerate, tocopherol, tocopherol acetate, avocado oil, camellia oil, turtle oil, macadamia nut oil, corn oil, mink oil, olive oil, rapeseed oil, egg yolk oil, sesame oil, persic oil, wheat germ oil, pasanqua oil, castor oil, linseed oil, safflower oil, cotton seed oil, perillic oil, soybean oil, peanut oil, tea seed oil, kaya oil, rice bran oil, china paulownia oil, Japanese paulownia oil, jojoba oil, rice germ oil, glycerol trioctanate, glycerol triisopalmiatate, trimethylolpropane triisostearate, glycerol tri-2-ethylhexanoate, pentaerythritol tetra-2-ethylhexanoate, lanolin, liquid lanolin, liquid paraffin, squalane, vaseline, and mixtures thereof. Commercially available oils include, for example, tridecyl isononanoate with tradename Crodamol TN available from Croda, Hexalan available from Nisshin Seiyu, and tocopherol acetates available from Eisai.

Non-volatile cosmetic emollients may include waxes such as, but not limited to paraffin wax, microcrystalline wax, ozokerite wax, ceresin wax, carnauba wax, candelilla wax, and eicosanyl behenate.

Non-volatile silicon oils may be used including, but not limited to polymethylphenylsiloxane, polydiphenylsiloxane, polydiethylsiloxane, polydimethylsiloxane (dimethicone). For purposes of the present disclosure, a non-volatile silicon oil is defined as one that has a kinematic viscosity greater than 10 centiStokes (cSt).

Suitable ancillary oils include polyalkyl or polyaryl siloxanes as disclosed in U.S. Pat. No. 6,936,241, the disclosure of which is incorporated by reference herein in its entirety.

Suitable ancillary oils useful herein include the various grades of mineral oils. Mineral oils are liquid mixtures of hydrocarbons that are obtained from petroleum. Specific examples of suitable hydrocarbons include paraffin oil, mineral oil, dodecane, isododecane, hexadecane, isohexadecane, eicosene, isoeicosene, tridecane, tetradecane, polybutene, polyisobutene, and mixtures thereof.

The non-volatile oil may not comprise a “volatile” silicone oil. A specifically excluded volatile silicone oil is decamethylcyclopentanasilaxane, commonly known as “D5.”

In the embodiments in which the substrate material is used in a non-cosmetic application such as in a consumer product as a coloring agent, a flavorant, a fragrance, or the like, the substrate material treated with the one or more cationic surface peptides also would be combined with conventional additives used in those formulations. The substrate material treated with the one or more cationic surface peptides has superior bio-adhesive properties and can adhere readily, and in a far improved manner when compared to un-treated substrate materials, to biological substrates such as nails, chitin, skin, hair, eyebrows, eyelashes, etc., without significant aggregation of the cosmetic material. Accordingly, when the substrate material is a coloring agent used in, for example, a hair dyeing composition or a shampoo, the substrate material would be combined with conventional dyeing composition or shampoo additives, carriers, and excipients. The coloring agent substrate treated with one or more cationic surface peptides would have superior adhesion to the hair when compared to the same coloring agent substrate that was not treated in accordance with the embodiments. Similarly, if the substrate material is a fragrance for use in, for example a deodorant, it would be used in combination with conventional deodorant additives and excipients and other components. The fragrance substrate material treated with one or more cationic surface peptides would have superior adhesion to skin, hair, nails and the like than the same fragrance substrate material that was not treated in accordance with the embodiments.

The foregoing description of the embodiments illustrates and describes the preferred embodiments but, as mentioned above, it is to be understood that the embodiments are capable of use in various other combinations, modifications, and environments and is capable of changes or modifications within the scope of the inventive concept as expressed herein, commensurate with the above teachings and/or the skill or knowledge of the relevant art. The embodiments described hereinabove are further intended to explain best modes known of practicing them and to enable others skilled in the art to utilize the embodiments in such, or other, embodiments and with the various modifications required by the particular applications or uses. Accordingly, the description is not intended to limit the embodiments to the form disclosed herein. Also, it is intended that the appended claims be construed to include alternative embodiments.

Throughout this application, various references including publications, patents, and pre-grant patent application publications are referred to. Disclosures of these publications in their entireties are hereby incorporated by reference into this application to more fully describe the state of the art to which the embodiments pertain. It is specifically not admitted that any such reference constitutes prior art against the present application or against any claims thereof. All publications, patents, and pre-grant patent application publications cited in this specification are herein incorporated by reference, and for any and all purposes, as if each individual publication or patent application were specifically and individually indicated to be incorporated by reference. In the case of inconsistencies the present disclosure will prevail.

The embodiments now will be explained in greater detail with reference to the following non-limiting examples.

### EXAMPLES

Preparation of Substrate Materials Treated with Poly-Lysine

Approximately 20-400 grams of water were mixed together with about 95-99 grams of silica beads (SOLESPHERE®, AGC Si-Tech Co., Ltd.), and about 0-0.2 grams of NaOH at about 50° C. using a ROBOMIX® disperser, commercially available from Primix Corporation, Osaka, Japan, for a period of about 20 minutes to form a homogenous substrate dispersion. To this homogenous substrate dispersion then were added about 4-10 grams of a solution containing 25% poly-lysine having the formula below, the poly-lysine present in an amount of about [Insert concentration] by weight.

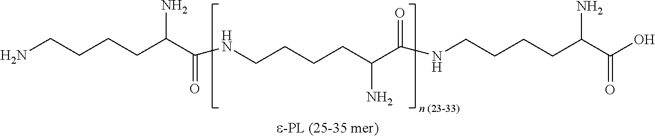

The mixture was again stirred with the above mixer at 5000 rpm for a period of 15 minutes to form a uniform homogenous dispersion of silica beads and poly-lysine. To this homogenized dispersion of silica beads and poly-lysine then were added stepwise by metering, an aluminum sulfate solution containing 0.04-1.50% by weight of aluminum sulfate, until the pH of the mixture reached a value of 4.0. The weight ratio of poly-lysine to aluminum sulfate was within the range of from 0.05 to 2.00. The product obtained then was separated from the mixture and dried at a temperature of about 105° C. for 16 hours to produce about silica beads having poly-lysine immobilized on the surface thereof.

This same process was repeated, except iron oxide powder was used instead of silica beads, thereby producing iron oxide powder having poly-lysine immobilized on the surface thereof.

### Comparative Example

The silica beads used in the example above were used without subjecting them to any treatment with poly-lysine, and hence were untreated silica beads.

### Hair Adhesion Test

The following Samples were used in this test: (a) the silica beads having poly-lysine immobilized on the surface thereof; (b) the iron oxide powder having poly-lysine immobilized to a surface thereof; and (c) the untreated silica beads. Approximately 0.25 g of each sample was mixed with about 0.25 g of cut hair in 50 ml of water, and mixed for about 10 minutes at 400 rpm. The water was filtered, and the resulting treated hair was washed with water, filtered again, and then dried at about 105° C. for 16 hours. Scanning electron photographs of the hair treated with (a), (b), and (c) were taken using a HITACHI TM-1000 at 1.0k magnification.

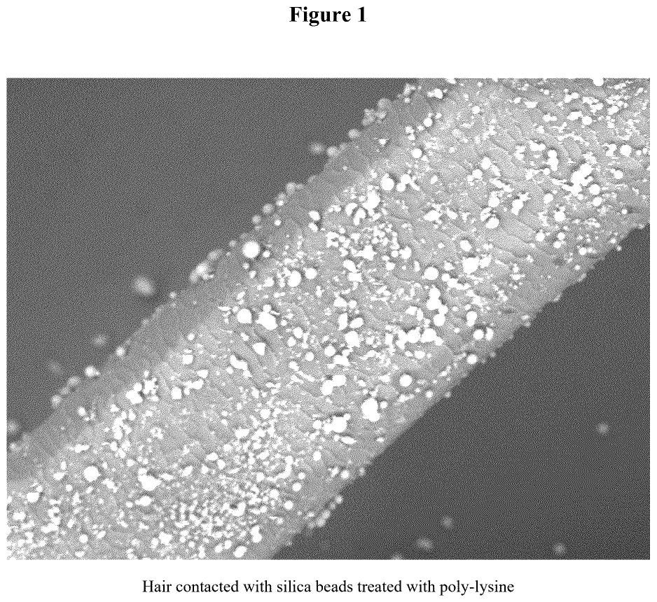

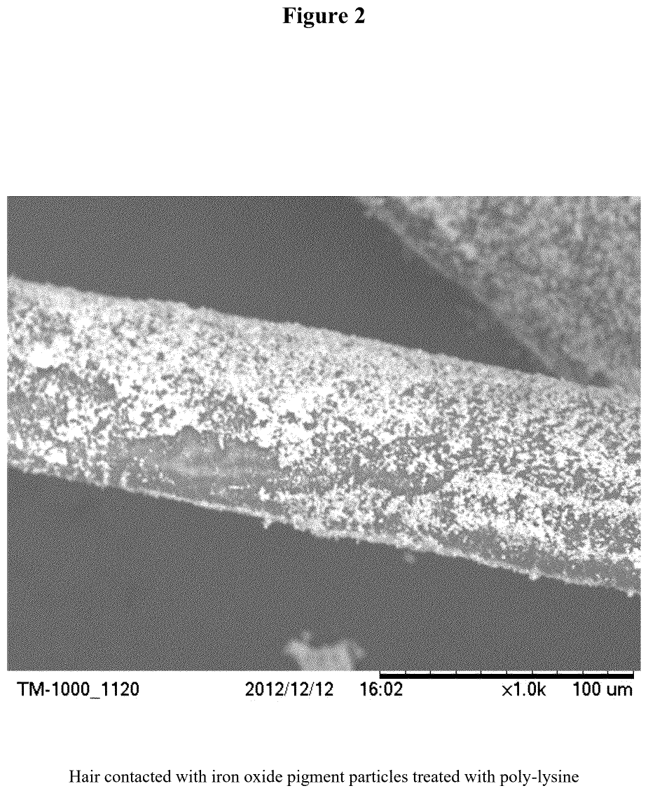

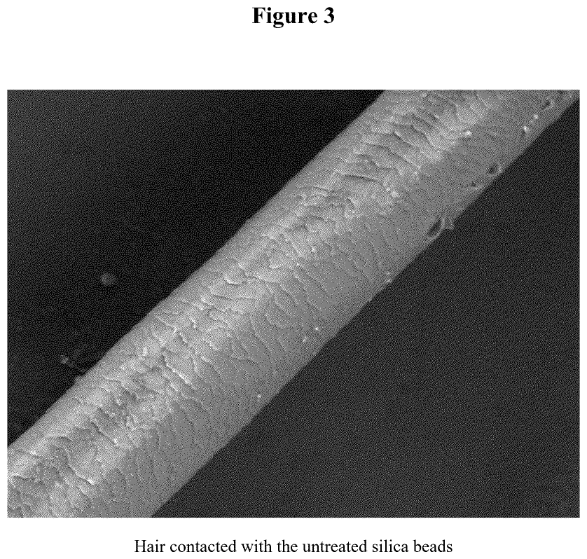

As shown in FIG. 1, the silica beads having poly-lysine immobilized on the surface thereof (Sample a) adhered well to the hair in a relatively uniform manner. As shown in FIG. 2, the iron oxide having poly-lysine immobilized on the surface thereof (Sample b) also adhered well and in a substantially uniform manner to the hair. In contrast, FIG. 3 shows that the untreated silica beads (Sample c) did not adhere at all to the hair.

The invention has been described with reference to particularly preferred embodiments. Those having ordinary skill in the art will readily appreciate that various modifications may be made to the invention without departing from the spirit and scope thereof.

1. A bioadhesive substance comprising at least one substrate material in which the surface of the at least one substrate material is chemically modified with at least one cationic surface peptide, wherein the at least one cationic surface peptide is chemically immobilized on the surface of at least one substrate material by a multivalent metal ion.

2. The bioadhesive substance of claim 1, wherein the at least one substrate material is a cosmetic material selected from the group consisting of inorganic pigment powders, organic pigment powders, powder substrates, extenders, extender pigments, and mixtures thereof.

3. The bioadhesive substance of claim 1, wherein the at least one substrate material comprises silica beads.

4. The bioadhesive substance of claim 1, wherein the at least one cationic surface peptide comprises a peptide having from 3 to 50 amino acids selected from the group consisting of arginine, lysine, and histidine.

5. The bioadhesive substance of claim 4, wherein the at least one cationic surface peptide comprises a peptide having from 5 to 40 lysine residues.

6. The bioadhesive substance of claim 5, wherein the at least one cationic surface peptide having from 5 to 40 lysine residues is represented by the following formula

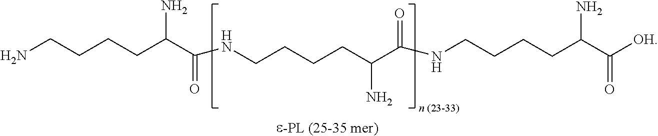

7. The bioadhesive substance of claim 1, wherein the at least one cationic surface peptide is present in an amount from about 0.05 to about 20% by weight, based on the weight of the at least one substrate material.

8. The bioadhesive substance of claim 7, wherein the at least one cationic surface peptide is present in an amount from about 0.1 to about 10% by weight, based on the weight of the at least one substrate material.

9. A method of making a surface-modified bioadhesive substance comprising:
(a) preparing an aqueous dispersion of at least one substrate material;
(b) adding to the aqueous dispersion at least one cationic surface peptide with agitation to uniformly disperse the mixture and form a substrate and peptide dispersion; and
(c) adding a multivalent metal-containing salt to neutralize the substrate and peptide dispersion and immobilize the at least one cationic surface peptide on the surface of the at least one substrate material.

10. The method of claim 9, wherein the multivalent metal-containing salt is aluminum sulfate.

11. The method of claim 9, wherein the multivalent metal-containing salt is added step-wise until the pH of the solution is within the range from about 3 to about 8.

12. The method of claim 11, wherein the multivalent metal-containing salt is added step-wise until the pH of the solution is about 4.0.

13. The method of claim 9, wherein the at least one substrate material is a cosmetic material selected from the group consisting of inorganic pigment powders, organic pigment powders, powder substrates, extenders, extender pigments, and mixtures thereof.

14. The method of claim 13, wherein the at least one substrate material comprises silica beads.

15. The method of claim 9, wherein the at least one cationic surface peptide, is present in an amount from about 0.05 to about 20 wt %, based on the weight of the at least one substrate material.

16. The method of claim 15, wherein the at least one cationic surface peptide comprises a peptide having from 3 to 50 amino acids selected from the group consisting of arginine, lysine, and histidine.

17. The method of claim 16, wherein the at least one cationic surface peptide is represented by the following formula

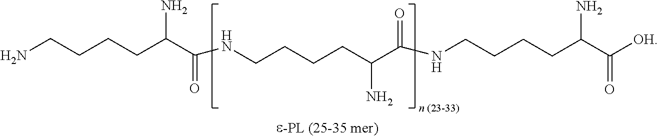

18. A cosmetic composition comprising:
(a) at least one bioadhesive substance comprising at least one substrate material in which the surface of the at least one substrate material is chemically modified with at least one cationic surface peptide, wherein the at least one cationic surface peptide is chemically immobilized on the surface of at least one substrate material by a multivalent metal ion; and
(b) a cosmetically acceptable carrier.

19. The cosmetic composition of claim 18, wherein the composition is in the form selected from the group consisting of a powder foundation, liquid foundation, point makeup, lip, mascara, eyeliner, skin care products which are skin cream, hair care products which are shampoo, conditioner, treatment and hair styling products, hair color products, cleansing products which are a body soap, hand soap and facial cleanser.

20. The cosmetic composition of claim 18, wherein the at least one substrate material is a cosmetic powder selected from the group consisting of inorganic pigment powders, organic pigment powders, powder substrates, extenders and extender pigments, or silica beads.
